(19) United States
(12) Patent Application Publication
(10) Pub. No.: US 2004/0158235 A1
(43) Pub. Date: Aug. 12, 2004

(54) TMR system and handpiece with ECG electrodes

(76) Inventors: Robert Rudko, Holliston, MA (US); David Hartung, Raynham, MA (US); Mark Tauscher, Medfield, MA (US)

Correspondence Address: Iandiorio & Teska, 260 Bear Hill Road, Waltham, MA, 02451-1018, US

(21) Appl. No.: 10/364,262

(22) Filed: Feb. 11, 2003

(51) Int. Cl. A61B 18/20

(52) U.S. Cl. 606/7; 606/12; 606/13; 606/19

(57) ABSTRACT

A TMR system including a handpiece with a barrel having a passage for transmitting a laser beam, a heart tissue contacting portion at one of the barrel, and at least one ECG electrode associated with the handpiece. An ECG unit is responsive to the ECG electrode for generating an ECG signal, a laser provides the laser beam, and a processing circuit is configured to fire the laser in response to the ECG signal.

### FIELD OF THE INVENTION

This invention relates to a transmyocardial revascularization system with a handpiece including ECG electrodes providing a stronger ECG signal to the TMR system.

### BACKGROUND OF THE INVENTION

In conventional transmyocardial revascularization (TMR) procedures, a patient is connected to an ECG via electrodes placed on his chest, his heart is exposed, and a laser is used to form channels in the wall of the heart. See U.S. Pat. No. 5,126,926 incorporated herein by this reference. The laser beam is delivered to a handpiece placed on the heart wall and the TMR system automatically triggers the laser to fire between the R and T waves of the heart's ECG cycle when the heart is relatively stable and electrically least sensitive to prevent fibrillation.

Thus, a clean and strong ECG signal is critical. But, since the heart is exposed during the TMR procedure, electrodes placed on the patient's chest do not always provide a strong enough ECG signal. Needle electrodes which pierce the patient's skin were also tried with somewhat better but not optimal results.

During the TMR procedure, any shortcomings or limitations of the TMR equipment can lengthen the procedure and/or frustrate the surgeon and hospital staff to the detriment of the patient.

### SUMMARY OF THE INVENTION

It is therefore an object of this invention to provide a TMR system and method with a stronger and cleaner ECG signal.

It is a further object of this invention to provide a safer TMR system.

It is a further object of this invention to provide a TMR handpiece with increased functionality.

The invention results, in part, from the realization that a stronger ECG signal is obtained by placing the ECG electrodes directly in contact with the heart itself and that if the TMR handpiece is modified to include the ECG electrodes, the handpiece now serves two functions: it is the laser beam delivery mechanism and it also provides the ECG signal to the TMR system for precise firing of the laser during the R and T waves of the heart beat cycle when the heart is relatively stable and electrically least sensitive to prevent fibrillation.

This invention features a TMR system comprising a handpiece including a barrel having a passage for transmitting a laser beam, a heart tissue contacting portion at one of the barrel, and at least one ECG electrode associated with the handpiece. An ECG is responsive to the ECG electrode for generating an ECG signal. A laser provides the laser beam and a processing circuit is configured to fire the laser in response to the ECG signal.

In the preferred embodiment, the electrode is biased into a position beyond the tissue contacting portion of the handpiece to ensure positive contact of the electrode with heart tissue. In one example, the electrode is integral with the tissue contacting portion of the handpiece. Preferably, the tissue contacting portion is a wall having a face with an aperture in communication with a passage in the barrel, the face extending continuously radially outward from the aperture to the periphery of the wall. The face may be knurled and the barrel straight or angled. Typically, the contacting wall is broader in cross-sectional area than the barrel and the contacting wall is flat with all of the edges rounded. In this example, the face may include at least one orifice therein, the ECG electrode located in the orifice. A spring typically biases the electrode outward from the face. Preferably, there are at least two electrodes integral with the face.

In another example, the electrode is disposed on an arm extending from the barrel portion of the handpiece. Typically, the arm includes a shoe housing the electrode. In one embodiment, the shoe extends beyond the tissue contacting portion of the handpiece and the arm is resilient to ensure positive contact of the electrode with heart tissue. Typically, there are two arms and two electrodes.

The preferred embodiment of the processing circuit includes a trigger pulse circuit configured to generate a trigger pulse having a width and a leading edge, a pulse positioning circuit configured to position the leading edge of the trigger pulse at a time during the contraction and expansion cycle of the heart beat which will not cause fibrillation of the heart, and a firing circuit configured to fire the laser at a time indicated by the trigger pulse position and for a period indicated by the width of the trigger pulse. Also, it is preferred that the processing circuit is further configured to inhibit actuation of the firing circuit in the absence of the ECG signal.

A combined handpiece/ECG electrode for transmyocardial revascularization in accordance with this invention features a barrel having a passage for transmitting a laser beam, a tissue contacting portion at one end of the barrel including an aperture in communication with the passage, and at least one ECG electrode associated with the handpiece.

A handpiece in accordance with this invention includes a barrel having a passage for transmitting a laser beam, a heart tissue contacting portion at one end of the barrel including an aperture in communication with the passage in the barrel, at least one ECG electrode associated with the handpiece, and an electrical conductor connected to the electrode, extending along the handpiece, and connectable to an ECG unit configured to generate an ECG signal.

One TMR system features a handpiece including a barrel having a passage for transmitting a laser beam and a heart tissue contacting portion at one of the barrel. At least one ECG electrode is engageable directly with the heart, an ECG is responsive to the ECG electrode for generating an ECG signal, a laser provides the laser beam, and a processing circuit is configured to fire the laser in response to the ECG signal. Typically, the ECG electrode is directly associated with the handpiece.

One method of making a combined handpiece/ECG electrode for transmyocardial revascularization in accordance with this invention features forming a barrel having a passage for transmitting a laser beam and a tissue contacting portion at one end of the barrel including an aperture in communication with the passage and associating at least one ECG electrode with the handpiece.

Preferably, the electrode is biased into a position beyond the tissue contacting portion of the handpiece to ensure positive contact of the electrode with heart tissue. In one example, the tissue contacting portion of the handpiece includes the electrode or electrodes. In another example, the electrode is disposed on an arm extending from the handpiece.

A method of firing a laser includes engaging an ECG electrode directly with heart tissue, generating an ECG signal based on the output from the ECG electrode, and firing a laser to form a channel in the heart tissue at a predetermined time during the ECG signal which will not cause fibrillation of the heart. Preferably, the ECG electrode is engaged directly with heart tissue via a handpiece which also delivers the laser beam to the heart tissue.

### BRIEF DESCRIPTION OF THE DRAWINGS

Other objects, features and advantages will occur to those skilled in the art from the following description of a preferred embodiment and the accompanying drawings, in which:

FIG. 1 is a schematic view of a prior art TMR system with an ECG unit connected to a patient via standard chest electrodes;

FIG. 2 is a block diagram showing one embodiment of a complete TMR system in accordance with the subject invention wherein the ECG signal is obtained from electrodes associated directly with the TMR handpiece;

FIG. 3 is a top view of one handpiece with integral ECG electrodes in accordance with the subject invention;

FIG. 4 is an end view showing one embodiment of the heart tissue contacting portion of the handpiece shown in FIG. 3;

FIG. 5 is a side view of the handpiece shown in FIG. 3;

FIG. 6 is a side view showing an angled barrel handpiece including electrodes in accordance with the subject invention;

FIG. 7 is a schematic three dimensional view showing another embodiment of a heart tissue contacting wall for the handpiece shown in FIG. 6;

FIG. 8 is a schematic cross-sectional view of one specific electrode/handpiece arrangement in accordance with the subject invention;

FIG. 9 is a schematic cross-sectional view showing another embodiment of a handpiece in accordance with the subject invention including ECG electrodes associated therewith;

FIG. 10 is a schematic partial three dimensional view showing in greater detail the electrode arms of FIG. 9; and

FIG. 11 is a schematic cross-sectional view showing one method of deriving the ECG signal directly from the patient's heart in accordance with this invention.

### DISCLOSURE OF THE PREFERRED EMBODIMENT

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings.

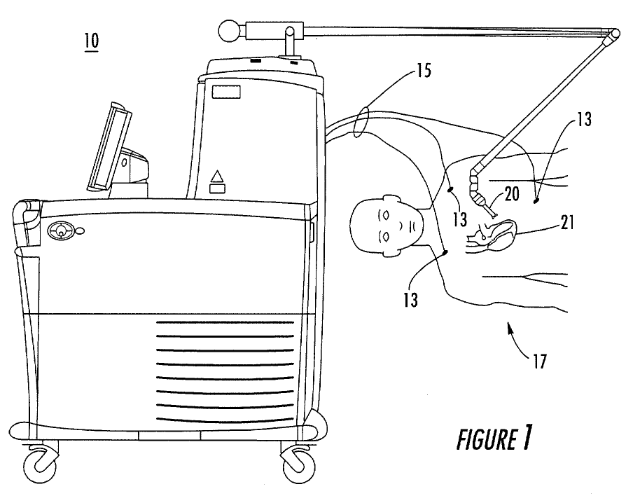

FIG. 1 shows prior art commercially successful TMR system 10 with the electrocardiogram unit thereof connected via electrodes 13 and wires 15 to patient 17. TMR system 10 then provides a laser beam through handpiece 20 placed on heart 21 only when the operating surgeon desires and automatically only between the R and T waves of the heart's ECG cycle when the heart is most stable and least sensitive electrically.

As delineated in the Background section above, since heart 21 is exposed, electrodes 13 placed on the patient's chest do not always provide a strong and clear signal to the ECG unit of TMR system 10. In response, needle electrodes inserted into the patient's chest were also tried, but not with optimal results.

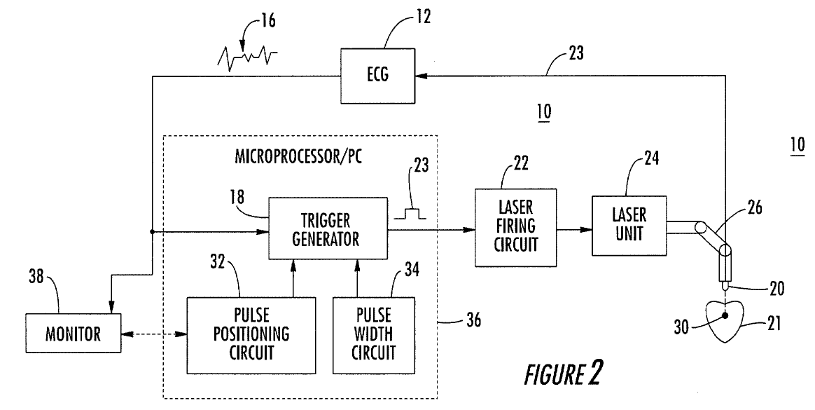

In the subject invention, in contrast, handpiece 20, FIG. 2 now serves to functions: it is the laser beam delivery mechanism and it also provides the ECG signal to TMR system 10 for precise firing of the laser beam during the R and T waves of the heart beat cycle when the heart is relatively stable and electrically least sensitive to prevent fibrillation. Handpiece 20 has at least one ECG electrode associated therewith connected to ECG unit 12 via wires 23. In this way, the ECG electrodes directly contact the wall of the heart for a stronger and clearer signal during the TMR procedure.

ECG signal 16 is delivered to trigger generator 18 which provides a trigger pulse 23 to laser firing circuit 22 which, in turn, energizes laser unit 24 with a laser power supply and a laser to produce a pulsed laser beam through articulated optical arm 26 into optical handpiece 20 to make a channel 30 in heart 21. The position of trigger pulse 23 in the heart beat cycle of ECG signal 16 is determined by pulse positioning circuit 32. The width of the pulse 23 and its duration during the heart beat cycle is determined by pulse width circuit 34. Trigger generator 18 as well as pulse positioning circuit 32 and pulse width circuit 34 may be included as an additional circuit board in a personal computer or as programming associated with microprocessor 36. Monitor 38 for ECG unit 12 typically displays both the ECG signal and information about trigger pulse 23. Additional details concerning the circuitry of the preferred TMR system of this invention are disclosed in U.S. Pat. No. 5,125,926.

Thus, the preferred processing circuitry of this invention may include trigger pulse circuit 18 configured to generate a trigger pulse having a width and a leading edge, pulse positioning circuit 32 configured to position the leading edge of trigger pulse 23 at a time during the contraction and expansion cycle of the heart beat which will not cause fibrillation of the heart, and firing circuit 22 configured to fire the laser to strike the beating heart in the time indicated by the trigger pulse position and for a period indicated by the width of the trigger pulse. As disclosed in the '926 patent, the processing circuitry of the TMR system is further configured to inhibit activation of firing circuit 22 in the absence of ECG signal 16. This safety feature ensures that the laser is only fired between the R and T waves of the heart beat cycle when the heart is relatively stable and electrically least sensitive to prevent fibrillation.

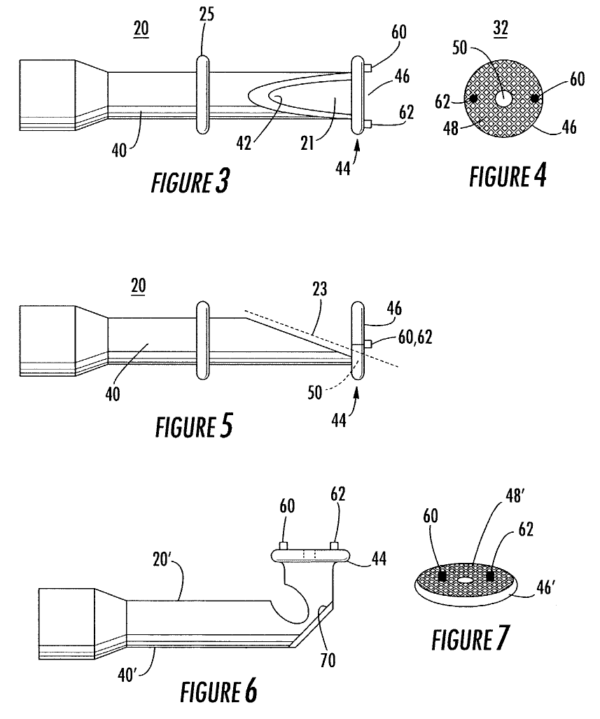

In one embodiment, handpiece 20, FIGS. 3-5 include barrel 40 having passage 42 therein for transmitting the laser beam and heart tissue contacting portion 44 at the distal end thereof. In this specific embodiment the tissue contacting portion is in the form of wall 46 having a face 48, FIG. 4 with aperture 50 therein in communication with passage 42 in barrel 40 and extending continuously radially outward from aperture 50 to the periphery of wall 46. Preferably, contacting wall 46 is broader in cross-sectional area than barrel 40 and contacting wall 46 is flat with all of the edges rounded. Barrel 40 may be straight or alternatively barrel 40′, FIG. 6 may be angled with mirror 70 therein to redirect the laser beam. In this embodiment, face 48′ of wall 46 has knurled ridges as shown. Tissue contacting face 48, FIG. 4 also has knurling as shown but in the form of cross-hatching or etching to prevent slippage when face 48 is in contact with the heart wall. See U.S. Pat. Nos. 6,132,422 and 6,133,587 incorporated herein by this reference.

ECG electrodes 60 and 62, in this embodiment, are integral with tissue contacting wall 46. In the embodiment shown in FIGS. 3-5, handpiece 20 also includes window 21 proximate contacting wall portion 44 for vapor release and also so that the surgeon can view the sight being lased. As shown in FIG. 5, the surgeon has a clear view of the lasing site proximate aperture 50 through window 21 along axis 23. Raised rim 25 on barrel 40 provides a finger grip for the surgeon to assist in gripping barrel 40.

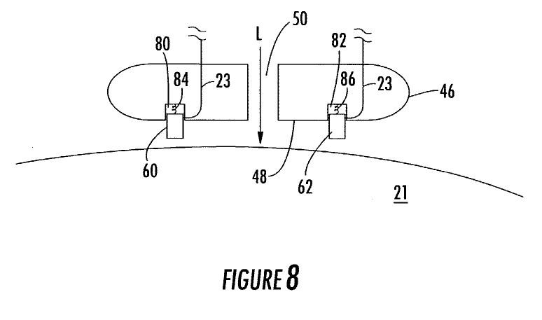

As shown in FIG. 8, face 48 may have one or more orifices 80, 82 therein for electrodes 60 and 62 and springs 84 and 86 may be included to bias electrodes 60 and 62 outward from face 48 to ensure positive contact of the electrodes with the heart 21 tissue when the surgeon positions wall face 48 on the heart. Wires 23 connected to each electrode extend within the handpiece and ultimately are connected to ECG unit 12, FIG. 2. In other embodiments, electrodes 60 and 62 and their connecting wires are simply molded directly into the handpiece. FIG. 8 depicts how the handpiece of this invention, in one preferred embodiment, serves as both the ECG sensing mechanism and the laser beam (L) delivery mechanism.

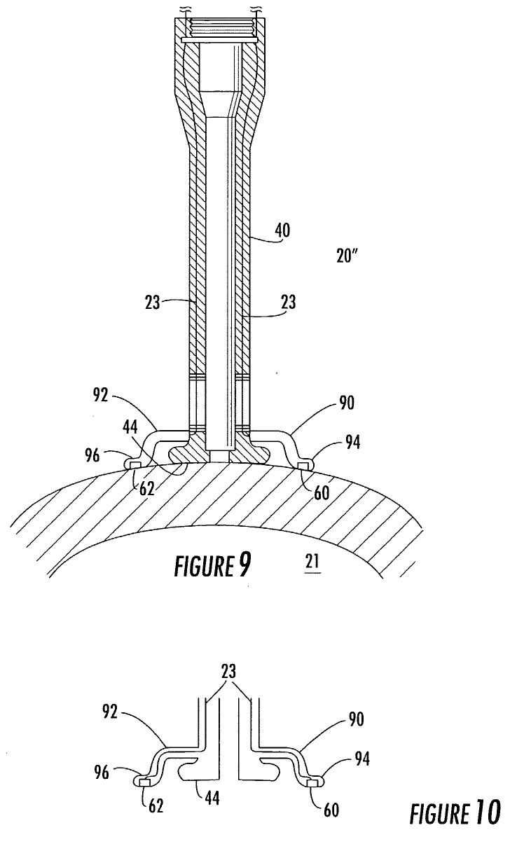

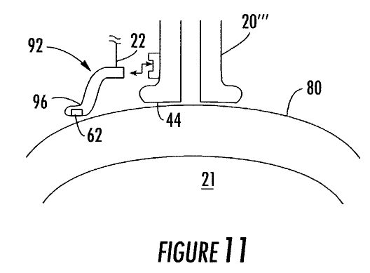

In another embodiment, handpiece 20″, FIG. 9 is fashioned with arms 90 and 92 extending from barrel portion 40 near tissue contacting wall 44 each terminating in shoes 94 and 96 housing electrodes 60 and 62. Preferably, shoes 94 and 96, FIG. 10 extend outward beyond tissue contacting wall 44 and arms 90 and 92 are made resilient to again ensure positive contact of the electrodes with the heart tissue. Typically, all of materials associated with the handpiece are electrically and thermally insulating except the electrodes.

Direct association of the ECG electrodes with the TMR handpiece is not a limitation of this invention, however. As shown in FIG. 11, electrode arm 92 may be detachable from handpiece 20′″ and clipped or otherwise secured to heart wall 80. In still other embodiments, other types of ECG electrode housings can be used to attach the ECG electrodes to the wall of the heart or a standard pacing electrode can be attached to the heart and used as an ECG electrode such as a Medtronic 6500 myocardial pacing lead connected to the ECG. In any embodiment, whether or not the ECG electrodes are a component of the handpiece, they are responsive directly to the heart for a cleaner and stronger signal.

In this way, the invention results in a TMR system with a stronger and cleaner ECG signal. In the specific embodiments of FIGS. 3-10, the TMR handpiece exhibits increased functionality and now provides two functions: it is the laser beam delivery mechanism and it also provides the ECG signal to the TMR system for precise firing of the laser beam between the R and T waves of the heart beat cycle when the heart is relatively stable and electrically least sensitive to prevent fibrillation.

One method of making a combined handpiece/ECG electrode for transmyocardial revascularization in accordance with this invention includes forming a barrel (e.g., barrel 40, FIG. 3) having a passage for transmitting a laser beam and tissue contacting portion 44 at one end of the barrel including an aperture in communication with the passage and associating at least one ECG electrode 60 with the hand piece. Preferably, as show in FIG. 8, the electrode is biased into a position beyond the tissue contacting portion of the handpiece to ensure positive contact of the electrode with heart tissue.

One method of firing a laser in accordance with this invention includes engaging an ECG electrode directly with heart tissue, generating an ECG signal based on the output from the ECG electrode, and firing a laser to form a channel in the heart tissue at a predetermined time during the ECG signal which will not cause fibrillation of the heart.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. For example, handpieces other than those shown in the drawings but fashioned with ECG electrodes may be used.

Other embodiments will occur to those skilled in the art and are within the following claims:

What is claimed is:

1. A TMR system comprising:
a handpiece including:
a barrel having a passage for transmitting a laser beam,
a heart tissue contacting portion at one of the barrel, and
at least one ECG electrode associated with the handpiece;
an ECG responsive to the ECG electrode for generating an ECG signal;
a laser for providing the laser beam; and
a processing circuit configured to fire the laser in response to the ECG signal.

2. The system of claim 1 in which the electrode is biased into a position beyond the tissue contacting portion of the handpiece to ensure positive contact of the electrode with heart tissue.

3. The system of claim I in which the electrode is integral with the tissue contacting portion of the handpiece.

4. The system of claim 3 in which the tissue contacting portion is a wall having a face with an aperture in communication with a passage in the barrel, the face extending continuously radially outward from the aperture to the periphery of the wall.

5. The system of claim 4 in which said face is knurled.

6. The system of claim 3 in which the barrel is straight.

7. The system of claim 4 in which the contacting wall is broader in cross-sectional area than the barrel and said contacting wall is flat with all of the edges rounded.

8. The system of claim 4 in which the face includes at least one orifice therein, the ECG electrode located in the orifice.

9. The system of claim 8 further including a spring which biases the electrode outward from the face.

10. The system of claim 3 in which there are at least two electrodes integral with the face.

11. The system of claim 1 in which the electrode is disposed on an arm extending from the handpiece.

12. The system of claim 11 in which the arm extends from the barrel portion.

13. The system of claim 11 in which the arm includes a shoe housing the electrode.

14. The system of claim 13 in which the shoe extends beyond the tissue contacting portion of the handpiece and the arm is resilient to ensure positive contact of the electrode with heart tissue.

15. The system of claim 11 in which the tissue contacting portion is a wall having a face with an aperture in communication with a passage in the barrel, the face extending continuously radially outward from the aperture to the periphery of the wall.

16. The system of claim 15 in which said face is knurled.

17. The system of claim 11 in which the barrel is straight.

18. The system of claim 11 in which the contacting wall is broader in cross-sectional area than the barrel and said contacting wall is flat with all of the edges rounded.

19. The system of claim 11 in which there are two arms and two electrodes.

20. The system of claim 1 in which the processing circuit includes:
a trigger pulse circuit configured to generate a trigger pulse having a width and a leading edge;
a pulse positioning circuit configured to position the leading edge of the trigger pulse at a time during the contraction and expansion cycle of the heart beat which will not cause fibrillation of the heart; and
a firing circuit configured to fire the laser at a time indicated by the trigger pulse position and for a period indicated by the width of the trigger pulse.

21. The system of claim 20 in which the processing circuit is further configured to inhibit actuation of the firing circuit in the absence of the ECG signal.

22. A combined handpiece/ECG electrode for transmyocardial revascularization comprising:
a barrel having a passage for transmitting a laser beam;
a tissue contacting portion at one end of the barrel including an aperture in communication with the passage; and
at least one ECG electrode associated with the handpiece.

23. The combined handpiece/ECG electrode of claim 22 in which the electrode is biased into a position beyond the tissue contacting portion of the handpiece to ensure positive contact of the electrode with heart tissue.

24. The combined handpiece/ECG electrode of claim 22 in which the electrode is integral with the tissue contacting portion of the handpiece.

25. The combined handpiece/ECG electrode of claim 24 in which the tissue contacting portion is a wall having a face with an aperture in communication with the passage in the barrel, the face extending continuously radially outward from the aperture to the periphery of the wall.

26. The combined handpiece/ECG electrode of claim 25 in which said face is knurled.

27. The combined handpiece/ECG electrode of claim 24 in which the barrel is straight.

28. The combined handpiece/ECG electrode of claim 25 in which the contacting wall is broader in cross-sectional area than the barrel and said contacting wall is flat with all of the edges rounded.

29. The combined handpiece/ECG electrode of claim 25 in which the face includes at least one orifice therein, the ECG electrode located in the orifice.

30. The combined handpiece/ECG electrode of claim 29 further including a spring which biases the electrode outward from the face.

31. The combined handpiece/ECG electrode of claim 25 in which there are at least two electrodes integral with the face.

32. The combined handpiece/ECG electrode of claim 22 in which the electrode is disposed on an arm extending from the handpiece.

33. The combined handpiece/ECG electrode of claim 32 in which the arm extends from the barrel portion.

34. The combined handpiece/ECG electrode of claim 32 in which the arm includes a shoe housing the electrode.

35. The combined handpiece/ECG electrode of claim 34 in which the shoe extends beyond the tissue contacting portion of the handpiece and the arm is resilient to ensure positive contact of the electrode with heart tissue.

36. The combined handpiece/ECG electrode of claim 32 in which the tissue contacting portion is a wall having a face with an aperture in communication with the passage in the barrel, the face extending continuously radially outward from the aperture to the periphery of the wall.

37. The combined handpiece/ECG electrode of claim 36 in which said face is knurled.

38. The combined handpiece/ECG electrode of claim 36 in which the barrel is straight.

39. The combined handpiece/ECG electrode of claim 36 in which the contacting wall is broader in cross-sectional area than the barrel and said contacting wall is flat with all of the edges rounded.

40. The combined handpiece/ECG electrode of claim 32 in which there are two arms and two electrodes.

41. A handpiece comprising:
a barrel having a passage for transmitting a laser beam;
a heart tissue contacting portion at one end of the barrel including an aperture in communication with the passage in the barrel;
at least one ECG electrode associated with the handpiece; and
an electrical conductor connected to the electrode, extending along the handpiece, and connectable to an ECG unit configured to generate an ECG signal.

42. A TMR system comprising:
a handpiece including:
a barrel having a passage for transmitting a laser beam, and
a heart tissue contacting portion at one of the barrel;
at least one ECG electrode engageable directly with the heart;
an ECG responsive to the ECG electrode for generating an ECG signal;
a laser for providing the laser beam; and
a processing circuit configured to fire the laser in response to the ECG signal.

43. The system of claim 42 in which the ECG electrode is associated with the handpiece.

44. The system of claim 42 in which the ECG electrode is a pacing lead.

45. A method of making a combined handpiece/ECG electrode for transmyocardial revascularization, the method comprising:
forming a barrel having a passage for transmitting a laser beam and a tissue contacting portion at one end of the barrel including an aperture in communication with the passage; and
associating at least one ECG electrode with the handpiece.

46. The method of claim 45 further including biasing the electrode into a position beyond the tissue contacting portion of the handpiece to ensure positive contact of the electrode with heart tissue.

47. The method of claim 45 in which the electrode is made integral with the tissue contacting portion of the handpiece.

48. The method of claim 45 in which the electrode is disposed on an arm extending from the handpiece.

49. A method of firing a laser comprising:
engaging an ECG electrode directly with heart tissue;
generating an ECG signal based on the output from the ECG electrode; and
firing a laser to form a channel in the heart tissue at a predetermined time during the ECG signal which will not cause fibrillation of the heart.

50. The method of claim 49 in which the ECG electrode is engaged directly with heart tissue via a handpiece which also delivers the laser beam to the heart tissue.

51. The method of claim 49 in which the electrode is a standard pacing electrode.
